(19) United States
(12) Patent Application Publication
(10) Pub. No.: US 2019/0381480 A1
(43) Pub. Date: Dec. 19, 2019

(54) A Separation Matrix and a Method of Separating Antibodies

(71) Applicant: GE Healthcare BioProcess R&D AB, Uppsala (SE)

(72) Inventors: Ronnie Palmgren, Uppsala (SE); Jean-Luc Maloisel, Uppsala (SE); Gustav Rodrigo, Uppsala (SE); Tomas Bjorkman, Uppsala (SE)

(21) Appl. No.: 16/486,653

(22) PCT Filed: Feb. 15, 2018
(86) PCT No.: PCT/EP2018/053816

§ 371 Date: Aug. 16, 2019

(30) Foreign Application Priority Data
Feb. 27, 2017 (GB) 1703116.2

(51) Int. Cl. B01J 20/286; B01D 15/38; B01J 20/28; B01J 20/32; B01D 15/18; C07K 1/22

(52) U.S. Cl. CPC B01J 20/286; B01D 15/3809; B01J 20/28004; B01J 20/28011; B01J 20/28019; B01J 2220/606; B01D 15/1807; B01D 15/1821; C07K 1/22; B01J 2220/52; B01J 20/3274

(57) ABSTRACT

A separation matrix comprising porous particles to which antibody-binding protein ligands have been covalently immobilized, wherein the density of said ligands is above 5 mg/ml, the volume-weighted median diameter of said porous particles is at least 10 and below 30 μm and the said porous particles have a gel phase distribution coefficient, expressed as K_{D} for dextran of molecular weight 110 kDa, of 0.5-0.9.

### TECHNICAL FIELD OF THE INVENTION

The present invention relates to separation matrices, and more particularly to a separation matrix useful in antibody separation. The invention also relates to a method of separating antibodies on the matrix.

### BACKGROUND OF THE INVENTION

In the manufacturing of therapeutic monoclonal antibodies (mAbs), affinity chromatography on matrices comprising coupled Staphylococcus Protein A (SpA) or variants of SpA is commonly used as a first separation step to remove most of the contaminants. As the demand for therapeutic mAbs is increasing there is a strong driving force for improving the efficiencies of the separation processes and several approaches are under evaluation.

Multicolumn continuous chromatography processes are available, where the feed is applied to a first column and is then diverted to one or more subsequent columns as the first columns approaches saturation and the first column is eluted and regenerated to be loaded again during elution and regeneration of the subsequent column(s). Such processes can be denoted periodic countercurrent chromatography (PCC) or simulated moving bed (SMB) and are of considerable interest for separation of therapeutic mAbs, see e.g. U.S. Pat. No. 7,901,581, US20130248451, US20130280788 and U.S. Pat. No. 7,220,356, which are hereby incorporated by reference in their entireties. PCC/SMB processes can significantly increase the productivity, but it appears that the full potential cannot be reached with currently available separation matrices, which are designed for conventional batch chromatography.

Accordingly, there is a need for new separation matrices specifically designed for continuous chromatography processes and for processes using such matrices. There is also a general need for separation matrices providing high dynamic binding capacities at very short residence times, in particular when used in shallow beds with low hydraulic resistance.

### SUMMARY OF THE INVENTION

One aspect of the invention is to provide a separation matrix allowing separation of mAbs in shallow beds with high productivity. This is achieved with a separation matrix comprising porous particles to which antibody-binding protein ligands have been covalently immobilized, wherein the density of said ligands is above 5 mg/ml and the volume-weighted median diameter of said porous particles is at least 10 and below 30 μm.

One advantage is that the matrix has a high binding capacity at very short residence times.

A second aspect of the invention is to provide a chromatography column allowing continuous separation of mAbs with high productivity. This is achieved with a column as defined in the claims.

A third aspect of the invention is to provide a multicolumn chromatography system allowing continuous separation of mAbs with high productivity. This is achieved with a system as defined in the claims.

A fourth aspect of the invention is to provide an efficient method of separating antibodies. This is achieved with a method as defined in the claims. One advantage is that the method allows very short residence times with high binding capacity.

Further suitable embodiments of the invention are described in the dependent claims.

### DRAWINGS

FIG. 1 shows an alignment of Protein A Fc-binding domains.

FIG. 2 shows the dynamic IgG-capacity vs residence time for prototype 128A compared with the 50 μm reference 871.

FIG. 2 shows the dynamic IgG-capacity vs residence time for prototypes 902B and 902A.

FIG. 4 shows a column according to the invention.

FIG. 5 shows a chromatography system according to the invention.

### DEFINITIONS

The terms “antibody” and “immunoglobulin” are used interchangeably herein, and are understood to include also fragments of antibodies, fusion proteins comprising antibodies or antibody fragments and conjugates comprising antibodies or antibody fragments.

The terms an “Fc-binding polypeptide” and “Fc-binding protein” mean a polypeptide or protein respectively, capable of binding to the crystallisable part (Fc) of an antibody and includes e.g. Protein A and Protein G, or any fragment or fusion protein thereof that has maintained said binding property.

The term “linker” herein means an element linking two polypeptide units, monomers or domains to each other in a multimer.

The term “spacer” herein means an element connecting a polypeptide or a polypeptide multimer to a support.

The term “% identity” with respect to comparisons of amino acid sequences is determined by standard alignment algorithms such as, for example, Basic Local Alignment Tool (BLAST™) described in Altshul et al. (1990) J. Mol. Biol., 215: 403-410. A web-based software for this is freely available from the US National Library of Medicine at http://blast.ncbi.nlm.nih.gov/Blast.cgi?PROGRAM=blastp&PAGE_TYPE=BlastSearch&LINK_LOC=blasthome. Here, the algorithm “blastp (protein-protein BLAST)” is used for alignment of a query sequence with a subject sequence and determining i.a. the % identity.

As used herein, the terms “comprises,” “comprising,” “containing,” “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### DETAILED DESCRIPTION OF EMBODIMENTS

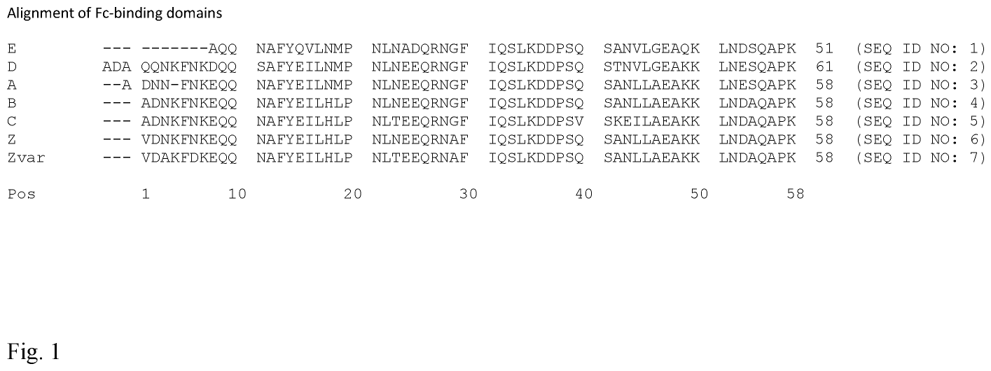

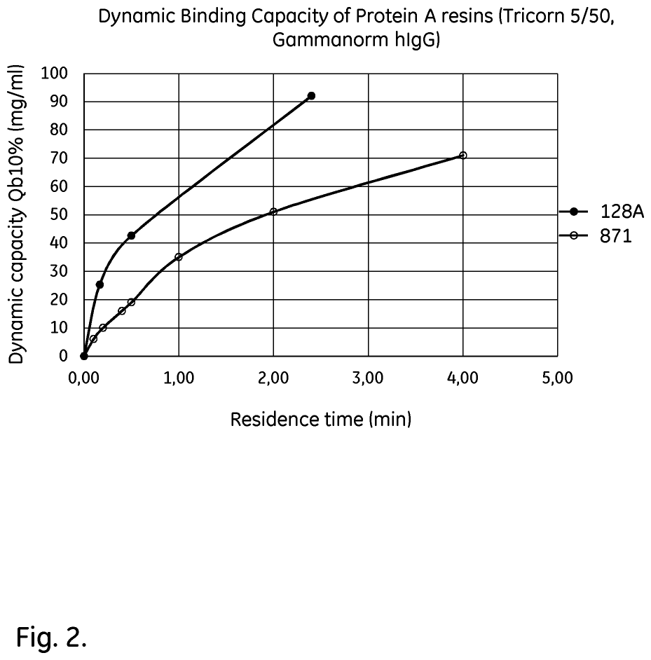

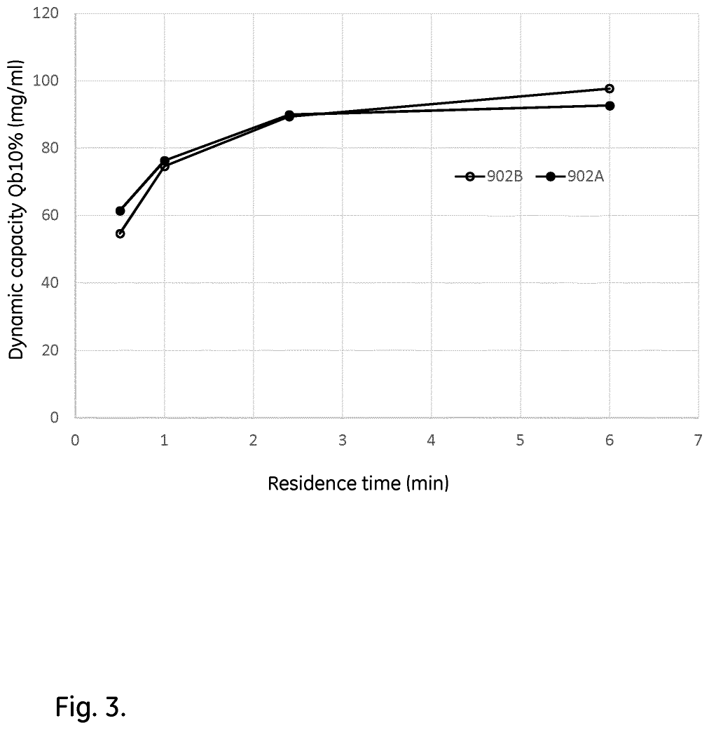

In one aspect, illustrated by FIGS. 1-3, the present invention discloses a separation matrix comprising porous, suitably spherical, particles to which antibody-binding protein ligands have been covalently immobilized. The density of these ligands is above 5 mg/ml, e.g. in the range of 5-25 mg/ml, such as 10.5-20 or 12-18 mg/ml, and the volume-weighted median diameter of the particles is at least 10 and below 30 μm, such as 10-29 μm or 15-28 μm. The density of the ligands denotes the content of coupled ligands per ml matrix sediment volume and it can be determined e.g. by amino acid analysis. The volume weighted median diameter, also denoted d50,v, can be determined by electrozone sensing (Coulter Counter), laser light diffraction or microscopy with image analysis. A preferred method is to use electrozone sensing with an instrument calibrated with a narrow sieve fraction of the matrix in question, for which the d50,v, has been determined with microscopy and image analysis.

The porous particles may comprise a crosslinked polysaccharide, which provides a large hydrophilic surface for coupling of the ligands, with minimal risk of non-specific interactions between mAbs or contaminants and the particles. The polysaccharide suitably has zero or very low (e.g. <5 micromol/ml) content of charged groups to prevent non-specific interactions. The crosslinking increases rigidity and chemical stability and can be achieved by methods known in the art, in particular by epoxide crosslinking, using e.g. epichlorohydrin or a diepoxide as crosslinker. Examples of polysaccharides can be dextran, cellulose and agarose. Agarose has the particular advantage that highly porous, rigid gels can be achieved by thermal gelation of aqueous agarose solution. The agarose can suitably be crosslinked by the methods described in U.S. Pat. Nos. 6,602,990, 7,396,467 or 8,309,709, which are hereby incorporated by reference in their entireties. Agarose crosslinked by these methods, so called high flow agarose, has a particularly advantageous combination of high rigidity and high porosity/pore volume, allowing high flow rates and rapid mass transport. High rigidity is particularly important for matrices having small particle sizes, to allow high flow rates without collapse of the matrix. The agarose can e.g. be allylated with reagents like allyl glycidyl ether or allyl halides before gelation, as described in U.S. Pat. No. 6,602,990. To allow for high binding capacities and rapid mass transport, the particles can advantageously have a large volume of pores accessible to macromolecular species like IgG antibodies. This can be determined by inverse size exclusion chromatography (SEC) as described in “Handbook of Process Chromatography, A Guide to Optimization, Scale-Up and Validation” (1997) Academic Press, San Diego, Gail Sofer & Lars Hagel eds. ISBN 0-12-654266-X, p. 368. A suitable parameter for the accessible pore volume is the gel phase distribution coefficient, K_{D}, determined for a probe molecule of defined size. This is a column-independent variable calculated from the retention volume V_{R} for the probe molecule, the interstitial void volume of the column V₀ and the total liquid volume of the column Vt according to K_{D}=(V_{R}−V₀)/(Vₜ−V₀). The porous particles can suitably have a K_{D} value in the range of 0.5-0.9, such as 0.6-0.85 or 0.65-0.85, for dextran of molecular weight 110 kDa as the probe molecule.

The ligands can e.g. be derived from antibody-binding bacterial proteins or derivatives thereof, such as SpA (Protein A), Peptostreptococcus Protein L or Streptococcus Protein G. Alternatively they can e.g. be derived from antibodies, such as single-chain camelid antibodies. They may bind to antibodies such that the affinity constant for the interaction is at most 1×10⁻⁶ M, for example at most 1×10⁻⁷ M, such as at most 1×10⁻⁹ M. They can comprise an Fc-binding protein, such as SpA or and SpA variant, which binds to the Fc part of IgG molecules. They can comprise monomers, dimers or multimers of native or mutated Protein A Fc-binding domains. The native Protein A Fc-binding domains E, D, A, B and C are shown in FIG. 1, together with the mutated variants Z and Zvar. In some embodiments, one or more of the domains in the ligands is derived from Protein Z or the B or C domain of Protein A, with the amino acid residue at position 23 being a threonine. Such domains have an improved alkali stability desirable for bioprocess use (see e.g. U.S. Pat. Nos. 8,329,860, 7,834,158, U.S. Ser. No. 14/961,164 and WO2016079033, hereby incorporated by reference in their entireties), which may e.g. be assessed by incubating the separation matrix 5 h in 0.5 M NaOH at 22+/−2° C. Suitably, the matrix after incubation retains at least 90% or at least 95% of the original IgG-binding capacity. In certain embodiments, one or more of the domains comprises an amino acid sequence as defined by SEQ ID NO: 8, 9 or 10, or having at least 90% such as at least 95% or at least 98% identity to SEQ ID NO: 8, 9 or 10, as calculated by the BLAST algorithm. SEQ ID NO:8 is the Zvar domain minus the linker sequence VDAKFD and SEQ ID NO:9 is the C domain minus the linker sequence ADNKFN. SEQ ID NO:10 is a further mutation of SEQ ID NO:8. One or more of the domains, such as all the domains, may also be further mutated by one or more amino acid substitutions, insertions or deletions. Thus, for example, there may be up to 10, 9, 8, 7, 6, 5, 4, 3 or 2 mutations, e.g. substitutions, within SEQ ID NO: 8, 9 or 10.

SEQ ID NO: 8
KEQQ NAFYEILHLP NLTEEQRNAF IQSLKDDPSQ SANLLAEAKK
LNDAQAPK
SEQ ID NO: 9
KEQQ NAFYEILHLP NLTEEQRNGF IQSLKDDPSV SKEILAEAKK
LNDAQAPK
SEQ ID NO: 10
KEAQ EAFYEILHLP NLTEEQRNAF IQSLKDDPSV SKAILAEAKK
LNDAQAPK

The ligands may comprise monomers, dimers or multimers of Protein A domains, typically wherein one or more of said domains have been mutated. One or more of the domains may e.g. be derived from Protein Z or the B or C domain of Protein A with the amino acid residue at position 23 being a threonine.

The ligands may additionally comprise one or more linker sequences of 1-10 amino acid residues, e.g. VDNKFN, ADNKFN, VDAKFD, AD or FN, suitably between the individual domains. In addition, the ligands may comprise a coupling moiety, e.g. a cysteine or a plurality of lysines at the C-terminus or N-terminus of the ligand, suitably at the C-terminus. The ligands may also comprise a leader sequence at the N-terminus, e.g. a scar or a residue after cleavage of a signal peptide and optionally also a copy of a linker sequence. Such a leader sequence may e.g. be a 1-15 amino acid (e.g. a 1-10 amino acid) peptide, e.g. AQ, AQGT, AQVDAKFD, AQGTVDAKFD or AQVDNKFN. Hence, a typical structure of a ligand may e.g. be Leader—(Domain-Linker)ₙ₋₁—Domain—Coupling moiety. n may e.g. be 1-7, such as 1, 2, 3, 4, 5, 6 or 7.

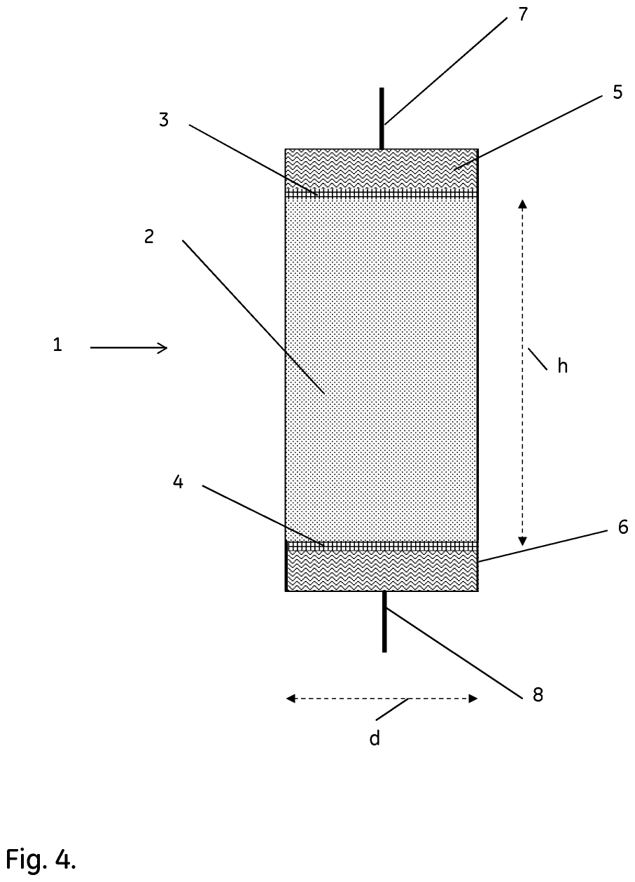

In a second aspect, illustrated by FIG. 4, the invention discloses a chromatography column 1 comprising the separation matrix as described above. The chromatography column can e.g. be an axial packed bed column 1, where a cylindrical packed bed 2 of matrix particles is confined between two nets/frits 3,4 and two distributor structures 5,6, allowing flow of a feed through an inlet 7, an inlet distributor 5 and an inlet net/frit 3 through the packed bed 2 and then through an outlet frit/net 4, an outlet distributor 6 and an outlet 8. The height h of the packed bed may e.g. be up to 5 cm or up to 10 cm, such as 2-5 cm, 2-4 cm or even 2-3 cm. The diameter d of the packed bed may e.g. be at least 0.5 or 1 cm, such as at least 1.5 cm or 1.5-200 cm, 1.5-100 cm, 1.5-50 cm or 1.5-30 cm. Suitably, the diameter/height ratio d/h may be >1, such as >2 or >3. This allows for reaching high capacities while keeping the back pressures low. The column can advantageously be a single use column, i.e. a pre-packed column constructed of low cost materials such as plastics, e.g. of polyolefins such as polypropylene and/or polyethylene. Scaling can be done by increasing the column diameter, thus increasing the d/h ratio as indicated above, but it is also possible to scale by using a plurality of columns coupled in parallel. Thus, the invention also discloses a plurality of columns as discussed above, coupled in parallel. Specific arrangements useful for parallel coupling of the columns are disclosed in e.g. US20120267299, US20130026100, US20130020263, US20133006867, US20140224738, US20140263012 and U.S. Ser. No. 15/329,199, which are hereby incorporated by reference in their entireties.

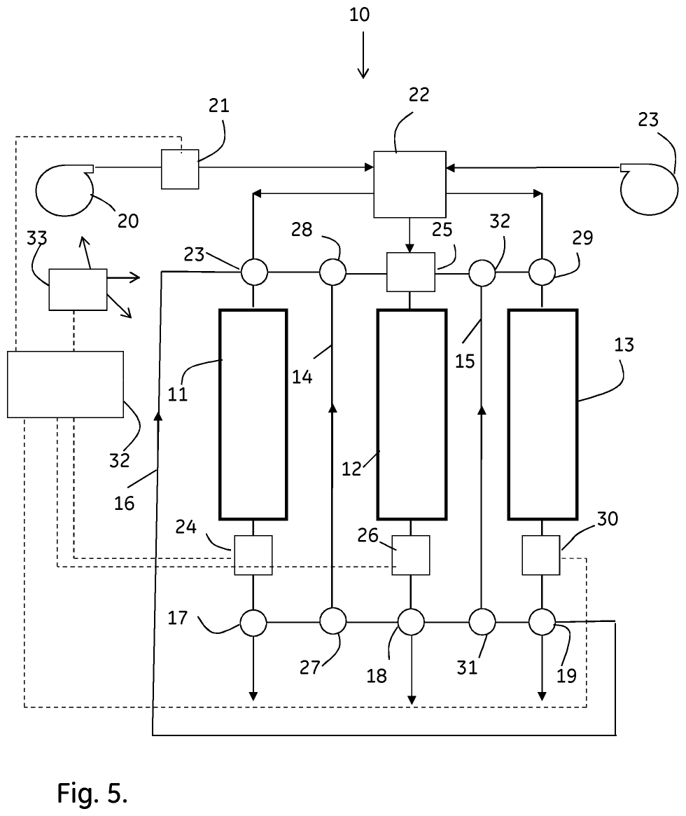

In a third aspect, illustrated by FIG. 5, the invention discloses a chromatography system 10 comprising a plurality of chromatography columns 11,12,13 as disclosed above (see also copending application PCT EP2016/069557, hereby incorporated by reference in its entirety). The system can suitably be arranged for performing continuous chromatography. It may e.g. comprise at least two, such as at least three chromatography columns 11,12,13 as disclosed above, packed with the same separation matrix and connected with one or more connecting lines 14,15,16 such that liquid can flow from one column 11,12 to a subsequent one 12,13 and from a last column 13 to a first column 11 and each connecting line between two columns may comprise at least one on/off valve 17,18,19, which may be three-way or four-way valves. The system may further comprise a feed pump 20, e.g. connected via a first detector 21 to a first valve block 22. A buffer pump 23 may also be connected to this first valve block 22. The first valve block 22 can further be connected to the inlet of a first column 11 via a first valve 23. An outlet end of the first column 11 may be connected to a second valve 17 through a second detector 24. The first valve block 22 can further be connected to the inlet of a second column 12 via a second valve or valve block 25. An outlet end of the second column 12 can be connected to valve 18 via a third detector 26. Furthermore, a valve 27 can be connected between valve 17 and valve 18. Valve 27 can also be connected to a valve 28 which is also connected to valve 23 and the second valve block 25. Hereby the effluent from the first column 11 can be directed to the inlet of the second column 12 through connecting line 14, valves 17, 27, 28 and 25. Furthermore the first valve block 22 can be connected to the inlet of a third column 13 via valve 29. An outlet end of the third column 13 may be connected to valve 19 via a fourth detector 30. Furthermore valve 31 can be connected between valve 18 and valve 19. Valve 31 can also be connected to valve 32 which may also be connected to the second valve block 25 and valve 29. Hereby the effluent from the second column 12 can be directed to the inlet of the third column 13 through connecting line 15. The effluent from the third column 13 can be directed to the inlet of the first column 11 through connecting line 16 via valves 19 and 23 (alternatively it can be directed to a subsequent fourth column). Furthermore, the first, second, third and fourth detectors 21, 24, 26, 30 may all be connected to a determining unit 32. The determining unit can be adapted to use the detected signals from the detectors to determine breakthrough and saturation points for the three different columns. The determining unit 32 and all the valve blocks, valves and pumps may further be connected to a control unit 33 (all the connections are not shown in the Figure) which is adapted to control the chromatography system in terms of when to remove or add columns from/into the loading zone, change flow rates, start new wash steps, etc. The detectors 21, 24, 26, 30 can e.g. be UV detectors. The control unit 33 may be configured to control the system according to breakthrough data obtained from the determining unit 32. Alternatively, control unit 33 can use fixed predetermined step times for the switching operations.

In a fourth aspect, the invention discloses a method of separation of antibodies by affinity chromatography. This method comprises the steps of:

a) conveying a process feed through at least a first chromatography column as disclosed above, to adsorb antibodies from the feed. The process feed may e.g. comprise at least 1-2 mg/ml or at least 4 mg/ml antibodies, such as 4-15, 4-10, or 4-5 mg/ml and/or the residence time in this step may e.g. be less than 2 min, such as 0.3-1 min or 0.3-0.8 min.;
b) optionally washing the first chromatography column;
c) conveying an eluent through the first chromatography column to elute antibodies; and
d) recovering the eluent with antibodies.

The method can suitably be carried out in the chromatography system 10 disclosed above. It can typically be carried out as a capture step, following clarification of a cell culture supernatant feed comprising the antibodies. After step d), the recovered eluent with antibodies may be subjected to further chromatography steps, such as ion exchange, multimodal chromatography and/or hydrophobic interaction chromatography steps.

In certain embodiments of the method, in step a) an effluent from the first chromatography column 11 is passed through a second chromatography column 12 packed with the same separation matrix as the first column;

after step a), in a step a′), the process feed is redirected to the second chromatography column 12 and an effluent from the second chromatography column is passed through a third chromatography column 13 packed with the same separation matrix as the first and second columns;
after step a′), in a step a″), the process feed is redirected to the third chromatography column 13 and an effluent from the third chromatography column is passed through the first chromatography column 11;
step c) is performed before step a″);
after step a′), in a step c′), the eluent is conveyed through the second chromatography column 12 to elute antibodies;
after step a″), in a step c″), the eluent is conveyed through the third chromatography column 13 to elute antibodies; and
the sequence of steps a), a′), a″), c), c′) and c″) is optionally repeated one or more times.

The residence time in steps a), a′) and a″) may e.g. be less than 2 min, such as 0.3-1 min or 0.3-0.8 min.

The method may further, after steps c), c′) and c″) respectively, comprise steps e), e′) and e″), each comprising conveying a cleaning liquid through said first, second and third chromatography columns respectively. The cleaning liquid can be an aqueous alkali solution comprising at least 0.1M (e.g. 0.1-1M or 0.1-0.5 M) alkali. 0.5-1 M alkali solutions can also be used as sanitizing solutions. The alkali may e.g. be NaOH, but can also be e.g. KOH. The cleaning (also called cleaning in place—CIP) step ensures that any residual feed components are removed from the columns before repetition of the binding and elution steps. Suitably, the ligands are capable of withstanding repeated alkali treatments, e.g. as discussed above where the matrix retains at least 95% of its original IgG-binding capacity after 5 h incubation with 0.5 M NaOH.

After steps e), e′) and e″) respectively, the method may also comprise equilibration steps f), f′) and f″) to reequlibrate the columns for steps a), a′) and a″) respectively.

### EXAMPLES

### Example 1

### Base Matrices

The base matrices used were a set of rigid cross-linked agarose bead sieve fraction samples of 17-50 micrometers (volume-weighted, d50V) median diameter (determined on a Malvern Mastersizer 2000 laser diffraction instrument), prepared according to the methods of U.S. Pat. No. 6,602,990 and with a pore size corresponding to an inverse gel filtration chromatography Kd value of 0.50-0.82 for dextran of Mw 110 kDa, according to the methods described in “Handbook of Process Chromatography, A Guide to Optimization, Scale-Up and Validation” (1997) Academic Press, San Diego, Gail Sofer & Lars Hagel eds. ISBN 0-12-654266-X, p. 368, using HR10/30 columns (GE Healthcare) packed with the prototypes in 0.2 M NaCl and with a range of dextran fractions as probe molecules (flow rate 0.2 ml/min). The base matrix prototypes were sieved to obtain desired particle size distributions.

TABLE 1
Base matrix prototypes

| Prototype | Kd (dextran 110 kDa) | d50v (μm) |
|---|---|---|
| 989 | 0.504 | 25 |
| 143 | 0.66 | 25 |
| 144 | | 25 |
| 743b | ~0.65 | 16.8 |
| 743a | ~0.65 | 27.7 |
| 965 | 0.823 | 25 |
| P14 | 0.732 | 21.2 |
| 178 | 0.65 | 50 |

### Coupling

100 ml base matrix was washed with 10 gel volumes distilled water on a glass filter. The gel was weighed (1 g=1 ml) and mixed with 30 ml distilled water and 8.08 g NaOH (0.202 mol) in a 250 ml flask with an agitator. The temperature was adjusted to 27+/−2° C. in a water bath. 16 ml epichlorohydrin (0.202 mol) was added under vigorous agitation (about 250 rpm) during 90+/−10 minutes. The reaction was allowed to continue for another 80+/−10 minutes and the gel was then washed with >10 gel volumes distilled water on a glass filter until neutral pH was reached. This activated gel was used directly for coupling as below.

To 16.4 mL of ligand solution (50 mg/mL) in a 50 ml Falcon tube, 139 mg NaHCO₃, 17.4 mg Na₂CO₃, 143.8 mg NaCl and 141 mg EDTA, was added. The Falcon tube was placed on a roller table for 5-10 min, and then 63 mg of DTE was added. Reduction proceeded for >45 min. The ligand solution was then desalted on a PD10 column packed with Sephadex G-25. The ligand content in the desalted solution was determined by measuring the 276 nm UV absorption.

The activated gel was washed with 3-5 GV {0.1 M phosphate/1 mM EDTA pH 8.6} and the ligand was then coupled according to the method described in U.S. Pat. No. 6,399,750 5.2.2, although with considerably higher ligand amounts (see below). All buffers used in the experiments had been degassed by nitrogen gas for at least 5-10 min. The ligand content of the gels was controlled by varying the amount and concentration of the ligand solution, adding 5-20 mg ligand per ml gel. The ligand was a tetramer of SEQ ID NO:8, with a VDAKFD linker and a C-terminal cysteine, except in prototypes 902A and B where it was a hexamer of SEQ ID NO:10 with a VDAKFD linker and a C-terminal cysteine.

After immobilization the gels were washed 3×GV (gel volumes) with distilled water. The gels+1 GV {0.1 M phosphate/1 mM EDTA/10% thioglycerol pH 8.6} was mixed and the tubes were left in a shaking table at room temperature overnight. The gels were then washed alternately with 3×GV {0.1 M TRIS/0.15 M NaCl pH 8.6} and 0.5 M HAc and then 8-10×GV with distilled water. Gel samples were sent to an external laboratory for amino acid analysis and the ligand content (mg/ml gel) was calculated from the total amino acid content. Table 1 shows the ligand contents of the coupled prototypes and the Kd and d50v data for the corresponding base matrices.

TABLE 1
Coupled prototypes

| | Kd | d50v | Ligand content |
|---|---|---|---|
| Prototype | (dextran 110 kDa) | (μm) | (mg/ml) |
| 129A | 0.504 | 25 | 7.1 |
| 129B | 0.504 | 25 | 12.4 |
| 128A | 0.66 | 25 | 7.0 |
| 128B | 0.66 | 25 | 11.1 |
| 124A | 0 | 25 | 6.9 |
| 124B | 0 | 25 | 11.6 |
| 796A | ~0.65 | 16.8 | 7.5 |
| 796B | ~0.65 | 16.8 | 13.1 |
| 795A | ~0.65 | 27.7 | 7.1 |
| 795B | ~0.65 | 27.7 | 12.2 |
| 118 | 0.823 | 25 | 6.6 |
| 902A | 0.732 | 21.2 | 15.4 |
| 902B | 0.732 | 21.2 | ~20 |
| 871 | 0.65 | 50 | 11 |

### Evaluation

The Qb10% dynamic capacity for polyclonal human IgG at 0.5, 1, 2.4 and 6 min residence time was determined as outlined below.

### Protein

Gammanorm 165 mg/ml (Octapharma), diluted to 2 mg/ml in Equilibration buffer.

### Equilibration Buffer

PBS Phosphate buffer 20 mM+0.15 M NaCl, pH 7.4

### Adsorption Buffer

PBS Phosphate buffer 20 mM+0.15 M NaCl, pH 7.4

### Elution Buffers

100 mM acetate pH 2.9

### Dynamic Binding Capacity

0.5 or 1 ml of resin was packed in TRICORN™ 5/50 columns (bed height 2.5-5 cm). The breakthrough capacity was determined with an ÄKTAExplorer 10 system at residence times of 0.5-6 minutes, with flow rates adjusted according to the residence time desired for the resin volume in question. Equilibration buffer was run through the bypass column until a stable baseline was obtained. This was done prior to auto zeroing. Sample was applied to the column until a 100% UV signal was obtained. Then, equilibration buffer was applied again until a stable baseline was obtained.

Sample was loaded onto the column until a UV signal of 85% of maximum absorbance was reached. The column was then washed with 5 column volumes (CV) equilibration buffer at flow rate 0.5 ml/min. The protein was eluted with 5 CV elution buffer at a flow rate of 0.5 ml/min. Then the column was cleaned with 0.5M NaOH at flow rate 0.2 ml/min and re-equilibrated with equilibration buffer.

For calculation of breakthrough capacity at 10% (q_{10%}), the equation below was used. q_{10%} is the amount of IgG that is loaded onto the column until the concentration of IgG in the column effluent is 10% of the IgG concentration in the feed.

$${q}_{10 \%} = \frac{{C}_{0}}{{V}_{C}} [ {V}_{app} - {V}_{sys} - {\int }_{{V}_{sys}}^{{V}_{app}} \frac{A ( V ) - {A}_{sub}}{{A}_{100 \%} - {A}_{sub}} ⋆ dv ]$$

A_{100%}=100% UV signal;

A_{sub}=absorbance contribution from non-binding IgG subclass;

A(V)=absorbance at a given applied volume;

V_{c}=column volume;

Vaₚₚ=volume applied until 10% breakthrough;

V_{sys}=system dead volume;

C₀=feed concentration.

The dynamic binding capacity (DBC) at 10% breakthrough was calculated. The dynamic binding capacity (DBC) was calculated for 10 and 80% breakthrough.

TABLE 2
Dynamic capacity results

| | | | Dynamic |
|---|---|---|---|
| | Bed height | Residence time | capacity, Qb10% |
| Prototype | (mm) | (min) | (mg/ml) |
| 129A | 25 | 0.5 | 31.6 |
| 129B | 27 | 0.5 | 21.2 |
| 128A | 25 | 0.5 | 42.6 |
| 128A | 25 | 2.4 | 92 |
| 128B | 26 | 0.5 | 47 |
| 128B | 26 | 2.4 | 84.2 |
| 124A | 24 | 0.5 | 41.4 |
| 124B | 26 | 0.5 | 32.8 |
| 796A | 25 | 0.5 | 54 |
| 796A | 25 | 1.0 | 59 |
| 796A | 25 | 2.4 | 62 |
| 796A | 25 | 6.0 | 70 |
| 796B | 25 | 0.5 | 61 |
| 796B | 25 | 1.0 | 71 |
| 796B | 25 | 2.4 | 81 |
| 796B | 25 | 6.0 | 101 |
| 795A | 25 | 1.0 | 49 |
| 795A | 25 | 2.4 | 55 |
| 795A | 25 | 6.0 | 61 |
| 795B | 25 | 0.5 | 38 |
| 795B | 25 | 1.0 | 55 |
| 795B | 25 | 2.4 | 67 |
| 795B | 25 | 6.0 | 77 |
| 118 | 50 | 0.5 | 41 |
| 118 | 50 | 1.0 | 46 |
| 118 | 50 | 2.4 | 52 |
| 902A | 50 | 0.5 | 54.7 |
| 902A | 50 | 1.0 | 74.6 |
| 902A | 50 | 2.4 | 89.4 |
| 902A | 50 | 6.0 | 97.7 |
| 902B | 50 | 0.5 | 61.4 |
| 902B | 50 | 1.0 | 76.2 |
| 902B | 50 | 2.4 | 90 |
| 902B | 50 | 6.0 | 92.7 |
| 871 | 50 | 0.5 | 19 |
| 871 | 50 | 1.0 | 35 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims. Any patents or patent applications mentioned in the text are hereby incorporated by reference in their entireties, as if they were individually incorporated.

1. A separation matrix comprising porous particles to which antibody-binding protein ligands have been covalently immobilized, wherein the density of said ligands is above 5 mg/ml, the volume-weighted median diameter of said porous particles is at least 10 and below 30 μm and the said porous particles have a gel phase distribution coefficient, expressed as KD for dextran of molecular weight 110 kDa, of 0.5-0.9.

2. The separation matrix of claim 1, wherein the density of said ligands is in the range of 5-25 mg/ml, such as 11-20 mg/ml.

3. The separation matrix of claim 1, wherein said porous particles have a gel phase distribution coefficient, expressed as KD for dextran of molecular weight 110 kDa, of 0.6-0.85.

4. The separation matrix of claim 1, wherein said porous particles are spherical.

5. The separation matrix of claim 1, wherein said porous particles comprise a crosslinked polysaccharide.

6. The separation matrix of claim 1, wherein said porous particles comprise crosslinked agarose.

7. The separation matrix of claim 6, wherein the agarose has been allylated before gelation.

8. The separation matrix of claim 1, wherein said ligands comprise an Fc-binding protein.

9. The separation matrix of claim 8, wherein said Fc-binding protein is Protein A.

10. The separation matrix of claim 1, wherein said ligands comprise monomers, dimers or multimers of Protein A domains.

11. The separation matrix of claim 10, wherein one or more of said domains have been mutated.

12. The separation matrix of claim 11, wherein one or more of said domains is derived from Protein Z or the B or C domain of Protein A and wherein the amino acid residue at position 23 is a threonine.

13. The separation matrix of claim 10, wherein one or more of said domains comprises an amino acid sequence as defined by, or having at least 90% or at least 95 or 98% identity to, SEQ ID NO: 8, 9 or 10.

14. The separation matrix of claim 1, which after 5 hours incubation in 0.5 M NaOH at 20+/−2° C. retains at least 95% of its original binding capacity.

15. The separation matrix of claim 8, wherein said matrix has a dynamic IgG capacity q10% of at least 20 mg/ml, such as at least 30 mg/ml at 0.5 min residence time.

16. A chromatography column (1) comprising the separation matrix according to claim 1.

17. The chromatography column of claim 16, comprising a packed bed of said separation matrix, wherein said packed bed has a bed height of up to 5 or 10 cm, such as 2-5 cm, 2-4 cm or 2-3 cm.

18. A chromatography system comprising a plurality of chromatography columns according to claim 16.

19. The chromatography system of claim 18, wherein said plurality of chromatography columns are coupled in parallel.

20. The chromatography system of claim 18, arranged for performing continuous chromatography.

21. The chromatography system of claim 18, comprising at least two, such as at least three, chromatography columns according to claim 16, packed with the same separation matrix and connected with one or more connecting lines such that liquid can flow from one column to a subsequent one and from a last column to a first column and wherein each connecting line between two columns comprises at least one on/off valve.

22. A method of separation of antibodies by affinity chromatography, which method comprises the steps of:
a) conveying a process feed through at least a first chromatography column according to claim 16, to adsorb antibodies from said feed;
b) optionally washing said first chromatography column;
c) conveying an eluent through said first chromatography column to elute antibodies; and
d) recovering said eluent with antibodies.

23. The method of claim 22, which is carried out in the chromatography system comprising a plurality of chromatography columns.

24. The method of claim 22, wherein:
in step a) an effluent from said first chromatography column is passed through a second chromatography column packed with the same separation matrix as the first column;
after step a), in a step a′), the process feed is redirected to the second chromatography column and an effluent from the second chromatography column is passed through a third chromatography column packed with the same separation matrix as the first and second columns;
after step a′), in a step a″), the process feed is redirected to the third chromatography column and an effluent from the third chromatography column is passed through the first chromatography column;
step c) is performed before step a″);
after step a′), in a step c′), the eluent is conveyed through the second chromatography column to elute antibodies;
after step a″), in a step c″), the eluent is conveyed through the third chromatography column to elute antibodies; and
the sequence of steps a), a′), a″), c), c′) and c″) is optionally repeated one or more times.

25. The method of claim 24, wherein in step a), the residence time is less than 2 min, such as 0.3-1 min or 0.3-0.8 min.

26. The method of claim 24, wherein in steps a), a′) and a″), the residence time is less than 2 min, such as 0.3-1 min or 0.3-0.8 min.

27. The method of claim 22, wherein said process feed comprises at least 4 mg/ml antibodies, such as 4-15 or 4-10 mg/ml.

28. The method of claim 24, further comprising steps e), e′) and e″), after steps c), c′) and c″) respectively, comprising conveying a cleaning liquid through said first, second and third chromatography columns.

29. The method of claim 28, wherein said cleaning liquid comprises at least 0.1 M alkali such as NaOH.


SEQUENCE LISTING (10 sequences)

SEQ ID NO: 1 - protein, 51 aa, Staphylococcus aureus
AQQNAFYQVLNMPNLNADQRNGFIQSLKDDPSQSANVLGEAQKLNDSQAPK

SEQ ID NO: 2 - protein, 61 aa, Staphylococcus aureus
ADAQQNKFNKDQQSAFYEILNMPNLNEEQRNGFIQSLKDDPSQSTNVLGEAKKLNESQAPK

SEQ ID NO: 3 - protein, 58 aa, Staphylococcus aureus
ADNNFNKEQQNAFYEILNMPNLNEEQRNGFIQSLKDDPSQSANLLAEAKKLNESQAPK

SEQ ID NO: 4 - protein, 58 aa, Staphylococcus aureus [sequence omitted]

SEQ ID NO: 5 - protein, 58 aa, Staphylococcus aureus
ADNKFNKEQQNAFYEILHLPNLTEEQRNGFIQSLKDDPSVSKEILAEAKKLNDAQAPK

SEQ ID NO: 6 - protein, 58 aa, Escherichia coli [sequence omitted]

SEQ ID NO: 7 - protein, 58 aa, Escherichia coli [sequence omitted]

SEQ ID NO: 8 - protein, 52 aa, Escherichia coli
KEQQNAFYEILHLPNLTEEQRNAFIQSLKDDPSQSANLLAEAKKLNDAQAPK

SEQ ID NO: 9 - protein, 52 aa, Escherichia coli
KEQQNAFYEILHLPNLTEEQRNGFIQSLKDDPSVSKEILAEAKKLNDAQAPK

SEQ ID NO: 10 - protein, 52 aa, Escherichia coli
KEAQEAFYEILHLPNLTEEQRNAFIQSLKDDPSVSKAILAEAKKLNDAQAPK